(19) United States
(12) Patent Application Publication
(10) Pub. No.: US 2016/0262926 A1
(43) Pub. Date: Sep. 15, 2016

(54) PORTABLE SEMICONDUCTOR APPARATUS FOR COLD AND HOT COMPRESS

(71) Applicant: Linyun Li, Ann Arbor, MI (US)

(72) Inventor: Linyun Li, Ann Arbor, MI (US)

(21) Appl. No.: 14/642,322

(22) Filed: Mar. 9, 2015

(51) Int. Cl. A61F 7/02; A61F 7/00

(52) U.S. Cl. CPC A61F 7/02; A61F 7/007; A61F 2007/0086; A61F 2007/0295; A61F 2007/0228

(57) ABSTRACT

This disclosure provides a portable semiconductor apparatus for treatment of an injury site in a body portion. The disclosure also provides for a method of treating the body portion. The apparatus comprises a fixing plate, a semiconductor element, a heat radiator, a fan element, and a temperature controller to change the apparatus between a hot mode and a cold mode. The apparatus further comprises adjustable straps, which secure the apparatus to the user. This portable apparatus may be used in hot and cold intervals to treat an injury remote from other first aid care.

### BACKGROUND

1. Technical Field

The present disclosure relates to medical devices. More particularly, the present disclosure relates to a portable semiconductor apparatus for treating an injury site in a body portion with a cold or hot compress.

2. Background Information

During physical activity, especially outdoor activity, individuals may experience accidental injuries. For example, playing sports may lead to muscle, joint, or bone injuries such as subcutaneous hemorrhage or edema. Typical treatment for such injury is a local hot or cold compress used promptly following the injury. Such prompt treatment may be important for minimizing injury and reducing recovery time. Ideally, such local hot or cold compress may be portable.

Current treatment options include using ice bags or evaporating refrigerants for local cold compresses. Such cold compresses may last for a limited time. In addition, semiconductor technology may be used for localized liquid cooling, as such disclosed in Chinese Patent CN86103453. However, these semiconductor devices may be difficult to carry around.

### BRIEF SUMMARY

The present disclosure provides generally for a portable semiconductor apparatus for treating a body portion with a cold or hot compress. The apparatus may include a fixing plate adapted to be disposed on the body portion and having a proximal side, a distal side, and a temperature sensor wherein the temperature sensor extends from the proximal side to the distal side.

The apparatus may further include a semiconductor element disposed distal to the distal side, the semiconductor element having a warm portion and a cold portion. A heat radiator may be disposed distal to the semiconductor element, a fan element may be disposed distal to the heat radiator, and a temperature controller may be in communication with the temperature sensor wherein the apparatus comprises a hot mode and a cold mode. The temperature controller may change the apparatus between the hot and cold modes to control the temperature.

The apparatus may further include a control circuit, a power supply, and a switch. The control circuit may be in electrical communication with the power supply and the switch, the control circuit being in communication with the temperature sensor. The switch may have a first position and a second position, the first position operating the control circuit in a first direction for the cold mode and the second position operating the control circuit in a second direction for the hot mode. In addition, the heat radiator may comprise a radiator end with the temperature controller attached to the radiator end. The temperature sensor may be adapted for thermal communication with the body portion.

In one embodiment, hot mode may be about 30 degrees Celsius to about 40 degrees Celsius. Additionally, cold mode may be about zero degrees Celsius to about 4 degrees Celsius. The semiconductor element may be inlaid in the distal side and adapted for thermal communication with the body portion. In one embodiment, the cold portion may be at or adjacent to the proximal side. In another embodiment, the warm portion may be at or adjacent to the proximal side.

The apparatus may further include a layer in communication with the semiconductor element and extending distally to the heat radiator, the layer being thermally conductive. In one embodiment, the layer may be silicone. Additionally, the apparatus may further have a plurality of fasteners that are attached to the fixing plate and extend distally through the heat radiator and fan element to secure the fixing plate, heat radiator, and fan element together.

The fixing plate may include a first fixing plate end, a second fixing plate end disposed opposite from the first fixing plate end, and a plurality of straps. The plurality of straps may be a first strap with a first connecting member and a second strap with a second connecting member. The first strap may be attached to the first fixing plate end and the second strap may be attached to the second fixing plate end. The first and second connecting members may be removably coupled to each other to secure the apparatus to the body portion. The first and second straps may be adjustable to fit the body portion.

The present disclosure also provides generally for a method of treating a body portion with the apparatus described in this disclosure. The method includes the steps of positioning a portable apparatus, setting the temperature controller to hot or cold mode, and operating the apparatus in hot or cold mode to treat the body portion. The step of operating the apparatus in cold mode may include operating the fan element to dissipate heat. In addition, the step of operating may include operating the apparatus in a hot interval and a cold interval to treat the body portion.

As one possible advantage, the ability to operate the portable apparatus in both hot and cold intervals, at different times, provides the user additional treatment options to treat an injury, particularly if the injury occurred in an area remote from other first aid care. Such hot and cold treatments are available with one apparatus.

The method may further include the steps of applying a medicant to the body portion before the step of positioning wherein the medicant is an anti-inflammatory or an analgesic agent. In addition, the method described herein may further comprise applying a wrap to the body portion after the step of applying a medicant and before the step of positioning.

Further objects, features and advantages of the invention will become readily apparent to persons skilled in the art after a review of the following description, with reference to the drawings and claims that are appended to and form a part of this specification.

### BRIEF DESCRIPTION OF THE DRAWINGS

FIG. 1 is an exploded side view of a portable apparatus for treating a body portion in accordance with one embodiment of the present invention;

FIG. 2 is a side view of the portable apparatus in FIG. 1;

FIG. 3 is a block diagram of a temperature controller in one embodiment of the portable apparatus in FIG. 1; and

FIG. 4 depicts the steps of one method of treating a body portion with the portable apparatus of FIG. 1 in accordance with one example of the present invention.

### DETAILED DESCRIPTION

The present disclosure provides a portable semiconductor apparatus for treatment of injury in a body portion of an individual. The disclosure also provides a method of treating a body portion with the apparatus. The materials, methods, and examples disclosed herein are illustrative only and not intended to be limiting. The disclosed figures are not necessarily to scale.

All publications, patent applications, patents, and other references mentioned herein are incorporated by reference in their entirety. Unless otherwise defined, all technical and scientific terms used herein have the same meaning as commonly understood by one of ordinary skill in the art to which this disclosure pertains. In case of conflict, the present document and definitions will control.

“Adjacent” referred to herein is at, nearby, near to, or in close proximity with.

The terms “proximal” and “distal” and derivatives thereof will be understood in the frame of reference of the individual using the apparatus; thus, proximal refers to locations closer to the individual and distal refers to locations further away from the individual.

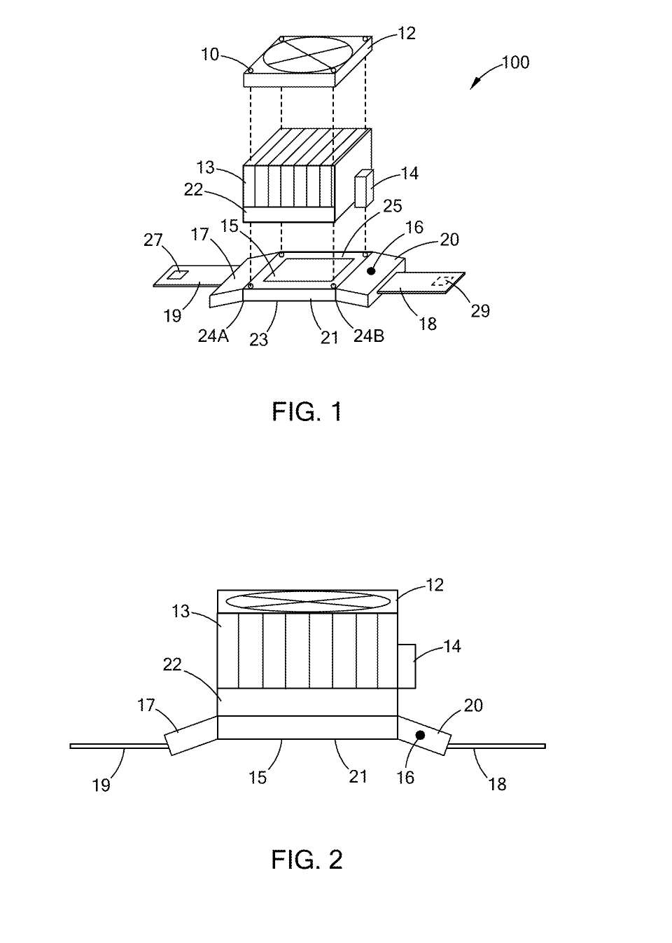

Referring now to the drawings, FIG. 1 illustrates the portable apparatus 100 in an exploded view. In this view, the fixing plate 21 is adapted for placement on the body portion. In FIG. 1, the fixing plate 21 comprises a proximal side 23 disposed about the body portion and a distal side 25 disposed away from the body portion. The fixing plate 21 may further comprise a temperature sensor 16 that extends from the proximal side 23 to the distal side 25, and is adapted for thermal communication with the body portion. In addition, the fixing plate 21 may further comprise a first fixing plate end 17 and a second fixing plate end 20 disposed opposite from the first fixing plate end 17.

In FIG. 1, the fixing plate 21 has a plurality of straps. A first strap 19 is attached to the first fixing plate end 17. A second strap 18 is attached to the second fixing plate end 20. The straps may extend horizontally from the apparatus in opposite directions. In one embodiment, the first strap 19 has a first connecting member 27. Likewise, the second strap 18 has a second connecting member 29.

The first and second connecting members (27 and 29, respectively) may be removably coupled to each other to secure the apparatus to the body portion. The first and second connecting members may be any mechanism known in the art to removably couple the straps to each other, such as snaps, tape, Velcro®, clips, pins, adhesive, and other such bonding. In one embodiment, the first and second straps (19 and 18, respectively) are adjustable to fit the body portion. This adjustability may be accomplished through any means known in the art, such as loops, slides, clips, and pins. In one embodiment, the straps may be sized and shaped to accommodate a leg or an arm. In another embodiment, the straps may be sized and shaped to accommodate a torso.

The fixing plate 21 may be made of any suitable material to those known in the art. The fixing plate may be a soft or flexible material to conform to a portion of the body. The first fixing plate end 17 and the second fixing plate end 20 connect to the fixing plate 21 through joints (24A and 24B, respectively). In one embodiment, one or both of these joints is a hinge to allow the first fixing plate end, fixing plate, and second fixing plate end to conform to the body portion. In addition, the first and second fixing plate ends (17 and 20, respectively) may also be made of a soft or flexible material.

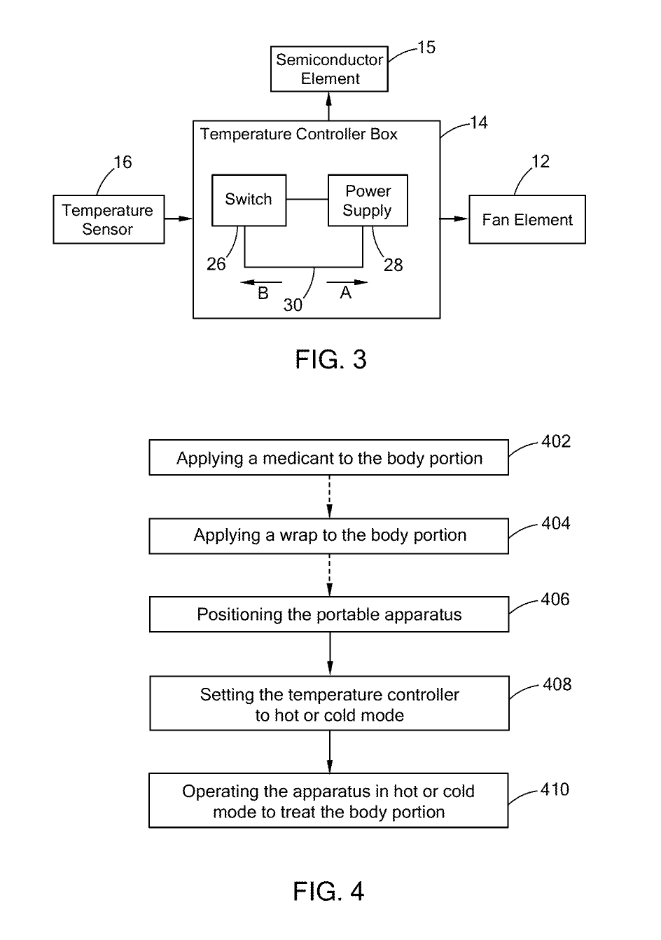

FIG. 1 illustrates a semiconductor element 15 disposed distal to the distal side 25 of the fixing plate 21. In one embodiment, the semiconductor element 15 is inlaid in or disposed within the distal side so it may be in thermal communication or direct contact with the body portion. The semiconductor element 15 comprises a warm portion and a cold portion. The warm portion and the cold portion may be created by direct electrical current supplied to the semiconductor element 15, as will be discussed further with FIG. 3. The location of the warm and cold portions may switch or alternate depending the flow of current.

In one embodiment, the semiconductor element may be an electronic heat pump or “Peltier” cell, which operates under the thermoelectric effect. One skilled in the art will understand that, under the thermoelectric effect, a difference in electrical voltage applied to each side of the Peltier cell may create a temperature difference across the cell on opposing sides. This temperature difference may be reversed by reversing the direction of the electrical current applied. This temperature difference may be used to create the warm and cold portion of the semiconductor element 15.

The semiconductor element may be formed from any material known in the art to create the warm and cold portions. For example, the semiconductor element may be formed from a semiconductive material doped with impurities.

A heat radiator 13 is disposed distal to the semiconductor element 15. The heat radiator 13 may be in direct contact with the semiconductor element 15. Alternatively, the heat radiator 13 may be separated from the semiconductor element 15 through a layer 22. The layer 22 may be in communication or contact with the semiconductor element 15 and may extend distally to the heat radiator 13. Preferably, the layer 22 is thermally conductive silicone.

With any arrangement, the heat radiator 13 may be disposed distal to the warm portion or the cold portion. If the heat radiator 13 or layer 22 contacts the cold portion, the heat radiator 13 may insulate the semiconductor element 15 and further warm the warm portion at or adjacent to the proximal side 23. If the heat radiator 13 or layer 22 contacts the warm portion, the heat radiator will conduct heat away from the semiconductor element 15 and further cool the cold portion at or adjacent to the proximal side 23.

The apparatus further comprises a fan element 12 disposed distal to or contacting the heat radiator 13. Preferably, the fan element is electric. The fan element may aid heat transfer away from the heat radiator 13. The apparatus is controlled through a temperature controller 14. The temperature controller 14 is in communication or contact with the temperature sensor 16. As necessary, the temperature controller 14 can change the apparatus between a hot mode and a cold mode to control the temperature at the injury site. The temperature controller 14 may be attached to the heat radiator 13 at a radiator end, as shown in FIG. 1.

The apparatus further comprises a plurality of fasteners 10. The fasteners 10 may be attached to the fixing plate 21, adjacent to the first and second fixing plate ends. The fasteners 10 may further extend distally from the fixing plate 21 through the heat radiator 13 and fan element 12 to secure the fixing plate 21, heat radiator 13, and fan element 12 together. The fasteners 10 may be any means known in the art. The fasteners 10 may be screws. (e.g. four screws at each corner of the apparatus).

FIG. 2 illustrates a side view of one embodiment of the apparatus. In this embodiment, the fixing plate 21 is directly attached or connected to the layer 22, the layer 22 is directly attached to the heat radiator 13, and the heat radiator 13 is directly attached to the fan element 12. It will be understood that these elements may be separated by other structures, features, or devices without falling beyond the scope and spirit of the invention.

FIG. 3 depicts further detail of the temperature controller 14. In this embodiment, the temperature controller 14 comprises a control circuit 30 being in electrical communication with a power supply 28 and a switch 26. As illustrated in FIG. 3, the temperature controller 14 is in communication with the temperature sensor 16, the semiconductor element 15, and the fan element 12. The temperature controller 14 may receive signal input from the temperature sensor 16 and give signal output to the fan element 12 and the semiconductor element 15. A skilled artisan will appreciate that the temperature controller 14 may be any means to receive signal input and give signal output, such as various electrical circuits including those operating with software to control inputs and outputs.

A skilled artisan will understand that the power supply 28 may be any source powerful enough to run the apparatus for a desired length of time. The power supply 28 may be a 12 Volt direct current source. Alternatively, the power supply 28 may be an alternating current power source or a non-portable power supply. Once the apparatus connects to the power supply 28, the user may operate it.

The temperature controller 14 operates the apparatus in either hot mode or cold mode. The switch 26 comprises a first position and a second position, the first position operates the control circuit 30 in a first direction A for the cold mode and the second position operates the control circuit 30 in a second direction B for the hot mode. The hot mode may be about 30 degrees Celsius to about 40 degrees Celsius. The cold mode may be about zero degrees Celsius to about 4 degrees Celsius.

In cold mode, the user may set the switch 26 to the first position. In this mode, the temperature sensor 16 may sense the temperature of the body portion or injury site and relay that signal to the control circuit 30. When the temperature is above about 4 degrees Celsius, the power supply 28 may generate a current that flows through the control circuit 30 in a first direction A. This flow of current creates the warm portion and the cold portion of the semiconductor element 15. In this embodiment, the cold portion would be in contact or close proximity with the body portion. The warm portion may contact the silicone layer.

As the heat radiator dissipates heat from the warm portion, the temperature controller 14 also may send a signal to the fan element 12 to spin or move. This movement further dissipates heat from the heat radiator. When the apparatus senses that the body portion is about zero degrees Celsius, the temperature controller 14 will shut of the apparatus until the temperature rises above about four degrees Celsius.

Alternatively in hot mode, the user may set the switch 26 to the second position. In this embodiment, the temperature sensor 16 may sense the temperature of the body portion or injury site and relay that signal to the control circuit 30. When the temperature is below about 30 degrees Celsius, the power supply 28 may generate a current that flows through the control circuit 30 in a second direction B. This flow of current creates the warm portion and the cold portion of the semiconductor element 15. In this embodiment, the warm portion could be in contact or close proximity with the body portion. The cold portion may be in contact with the silicone layer or heat radiator. When the apparatus senses that the body portion is about 40 degrees Celsius, the temperature controller 14 will shut of the apparatus until the temperature cools below about 30 degrees Celsius.

Preferably, the apparatus is small enough to be easily portable so that the user may take this apparatus with him or her while engaging in outdoor activities or other places where the user may sustain an injury remote from other available first aid or care. For example, the user would use the apparatus outdoors until he or she can get to an indoor location to receive other treatment.

FIG. 4 depicts a flow diagram of treating a body portion. Steps 402 and 404, shown with dotted arrows, are optional steps to be performed if desired. In step 402, the user may apply a medicant to the body portion. The medicant may be an anti-inflammatory or an analgesic agent. In step 404, the user may apply a wrap to the body portion. The wrap may be applied over the medicant.

In step 406, the user may position the portable apparatus, as described above, on the body portion. In step 408, the user may set the temperature controller to hot or cold mode. In step 410, the user may operate the apparatus in hot or cold mode, as described above, to treat the body portion. Optionally, the step of operating the apparatus in cold mode may comprise operating the fan element to dissipate heat. In addition, the step of operating may comprise operating the apparatus in a hot interval and a cold interval.

This may involve, first, running the apparatus in the hot interval, in hot mode as described herein. In addition, this may involve, second, running the apparatus in the cold interval, in the cold mode as described herein. Alternatively, these intervals could be reversed with the cold interval first and the hot interval second until the individual or user no longer desires treatment. There may be one or multiple hot and cold intervals to treat the injury site as desired by the user.

While the present invention has been described in terms of certain preferred embodiments it will be understood that the invention is not limited to this disclosed embodiments as those having skill in the art may make various modifications without departing from the scope of the following claims and equivalents thereof.

1. A portable apparatus for treating a body portion, the body portion having a temperature, comprising:
a fixing plate adapted to be disposed on the body portion and comprising a proximal side, a distal side, and a temperature sensor wherein the temperature sensor extends from the proximal side to the distal side;
a semiconductor element disposed distal to the distal side, the semiconductor element comprising a warm portion and a cold portion;
a heat radiator disposed distal to the semiconductor element;
a fan element disposed distal to the heat radiator; and
a temperature controller in communication with the temperature sensor wherein the apparatus comprises a hot mode and a cold mode and the temperature controller changes the apparatus between the hot and cold modes to control the temperature.

2. The apparatus of claim 1 wherein the temperature controller comprises a control circuit, a power supply, and a switch, the control circuit being in electrical communication with the power supply and the switch, the control circuit in communication with the temperature sensor.

3. The apparatus of claim 2 wherein the switch comprises a first position and a second position, the first position operating the control circuit in a first direction for the cold mode and the second position operating the control circuit in a second direction for the hot mode.

4. The apparatus of claim 3 wherein the heat radiator comprises a radiator end and the temperature controller is attached to the radiator end.

5. The apparatus of claim 1 wherein the temperature sensor is adapted for thermal communication with the body portion.

6. The apparatus of claim 1 wherein the hot mode is about 30 degrees Celsius to about 40 degrees Celsius.

7. The apparatus of claim 1 wherein the cold mode is about zero degrees Celsius to about 4 degrees Celsius.

8. The apparatus of claim 1 wherein the semiconductor element is inlaid in the distal side.

9. The apparatus of claim 8 wherein the semiconductor element is adapted for thermal communication with the body portion.

10. The apparatus of claim 1 further comprising a layer in communication with the semiconductor element and extending distally to the heat radiator, the layer being thermally conductive.

11. The apparatus of claim 10 wherein the layer comprises silicone.

12. The apparatus of claim 1 further comprising a plurality of fasteners that are attached to the fixing plate and extend distally through the heat radiator and fan element to secure the fixing plate, heat radiator, and fan element together.

13. The apparatus of claim 1 wherein the fixing plate comprises a first fixing plate end, a second fixing plate end disposed opposite from the first fixing plate end, and a plurality of straps.

14. The apparatus of claim 13 wherein the plurality of straps is a first strap with a first connecting member and a second strap with a second connecting member, the first strap being attached to the first fixing plate end and the second strap being attached to the second fixing plate end, the first and second connecting members being removably coupled to each other to secure the apparatus to the body portion.

15. The apparatus of claim 14 wherein the first and second straps are adjustable to fit the body portion.

16. The apparatus of claim 1 wherein the cold portion is at or adjacent to the proximal side.

17. The apparatus of claim 1 wherein the warm portion is at or adjacent to the proximal side.

18. A method for treating a body portion, the body portion having a temperature, the method comprising:
positioning a portable apparatus, the apparatus comprising:
a fixing plate adapted to be disposed on the body portion and comprising a proximal side, a distal side, and a temperature sensor wherein the temperature sensor extends from the proximal side to the distal side;
a semiconductor element disposed distal to the distal side, the semiconductor element comprising a warm portion and a cold portion;
a heat radiator disposed distal to the semiconductor element;
a fan element disposed distal to the heat radiator; and
a temperature controller in communication with the temperature sensor wherein the apparatus comprises a hot mode and a cold mode and the temperature controller changes the apparatus between the hot and cold modes to control the temperature;
setting the temperature controller to hot or cold mode; and
operating the apparatus in hot or cold mode to treat the body portion.

19. The method of claim 18 wherein the step of operating the apparatus in cold mode comprises operating the fan element to dissipate heat.

20. The method of claim 18 wherein the step of operating the apparatus comprises operating the apparatus in a hot interval and a cold interval.
